(19) United States
(12) Patent Application Publication
(10) Pub. No.: US 2007/0270973 A1
(43) Pub. Date: Nov. 22, 2007

(54) APPARATUS AND METHOD FOR SCULPTING THE SURFACE OF A JOINT

(75) Inventors: Wesley D. Johnson, Eden Prairie, MN (US); Daryl R. Pilarski, East Bethel, MN (US); Aaron Bisek, Elk River, MN (US); Robert Kohler, Lake Elmo, MN (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: ALEXANDRIA RESEARCH TECHNOLOGIES, LLC, Plymouth, MN (US)

(21) Appl. No.: 11/733,662

(22) Filed: Apr. 10, 2007

Related U.S. Application Data
(60) Provisional application No. 60/744,535, filed on Apr. 10, 2006.

(51) Int. Cl. A61F 2/44

(52) U.S. Cl. 623/17.16; 606/99

(57) ABSTRACT

A method and apparatus for minimally invasive total joint replacement. The method involves sculpting the articular surface of a second bone that normally articulates with a first bone by attaching a bone sculpting tool directly or indirectly to the first bone with the tool in bone sculpting engagement with the articular surface of the second bone, and then sculpting the articular surface of the second bone with the joint reduced and moving one bone with respect to the other. An implant is placed to replace the articular surface of the second bone using an impaction device directly or indirectly attached to the first bone.

### CROSS REFERENCE TO RELATED APPLICATIONS

This application claims the benefit of U.S. Patent Application Ser. No. 60/744,535, filed Apr. 10, 2006, the entirety of which is hereby incorporated by reference.

### THE FIELD OF THE INVENTION

The present invention is directed generally to the field of hip arthroplasty. This invention relates generally to surgical instruments and more particularly to an apparatus and method for implanting prostheses during surgery. The invention is specifically directed to an improved acetabular impactor uniquely constructed for use in less and minimally invasive hip surgeries.

### BACKGROUND OF THE INVENTION

A joint generally consists of two relatively rigid bony structures that maintain a relationship with each other. Soft tissue structures spanning the bony structures hold the bony structures together and aid in defining the motion of one bony structure relative to the other. In the hip, for example, the bony structures are the pelvis and the femur. Soft tissue such as ligaments, tendons and capsule span the joint and provide stability. A smooth and resilient surface consisting of articular cartilage covers the articulating structures. The articular surfaces of the bony structures work in concert with the soft tissue structures to form a mechanism that defines the envelope of motion between the structures. When the joint is taken through a full range of motion, the motion defines a total envelope of motion between the bony structures. Within a typical envelope of motion, the bony structures move in a predetermined pattern with respect to one another. In the example of the hip joint, the joint is a ball in socket joint that is inherently stable. The capsule and ligaments spanning the hip joint provide stability while the muscles provide motion.

Degenerative arthritis causes progressive pain, swelling, and stiffness of the joints. As the arthritis progresses the joint surfaces wear away and progression of the disease process increases pain and reduces mobility. Treatment of the afflicted articular bone surfaces depends, among other things, upon the severity of the damage to the articular surface and the age and general physical robustness of the patient. Commonly, for advanced arthritis, joint replacement surgery is necessary wherein the articulating elements of the joint are replaced with artificial elements commonly consisting of a part made of metal articulating with a part made of ultra high molecular weight polyethylene (UHMWPE). More recently, metal on metal and ceramic on ceramic bearing surfaces have gained in popularity. Early techniques for performing total joint arthroplasty involved large incisions and surgical exposures. Excessive trauma to soft tissue structures leads to significant intraoperative blood loss, postoperative pain, prolonged hospital stay, and slower recovery. The exposure must be sufficient to permit the introduction of drills, reamers, broaches and other instruments for cutting or removing cartilage and bone that subsequently is replaced with artificial surfaces.

For total hip replacement, the acetabular articular surface and subchondral bone are removed by hemi-spherical reamers. The femoral head is resected with an oscillating saw, the femoral canal may be prepared with reamers and the proximal medullary canal is shaped with broaches. Traditionally, the acetabulum is prepared with hemi-spherical reamers supported on straight drive handles and powered by a surgical drill. Extensive surgical exposure is needed to properly orient the acetabular reamer relative to the acetabulum. This has resulted in a need for instruments that take maximum advantage of available space.

Examples of instruments specifically described as being designed for minimally invasive hip surgery are shown in, for example, U.S. Pub. 2004/0153063 (Harris), U.S. Pat. No. 7,004,946 (Parker et al), U.S. Pat. No. 7,037,310 (Murphy), and U.S. Pub. 2006/0149285 (Burgi et al). While these devices may be acceptable for their intended purposes or described uses, each requires displacement of the femur to some extent to place the impactor handle and to impact the acetabular shell.

For patients who require hip replacement surgery it is desirable to provide surgical methods and apparatuses that enable preparation of implant support surfaces and implant placement without substantial damage or trauma to associated muscles, ligaments or tendons. Such minimally invasive total hip surgery reduces exposure of the joint cavity, and the size and location of the minimally invasive incision may not be optimal for proper orientation and application of force to adequately seat and stabilize an acetabular implant. Thus, an impaction device is needed that allows for impaction of the acetabular component with the hip reduced or articulated for use with a minimally invasive exposure for total hip arthroplasty. It may also be desirable to use an alignment guide or surgical navigation to aid the surgeon in positioning the acetabular implant. To attain this goal, a system and method is needed to enable articulating surfaces of the joints to be appropriately sculpted and implants to be placed using minimally invasive apparatuses and procedures. What is needed is an acetabular cup impactor that is more easily placed into the joint space, maintains the femur in an anatomical position and enables cup impaction.

### SUMMARY OF THE INVENTION

The present invention provides an apparatus and method for acetabular cup impaction during hip arthroplasty involving minimally invasive surgical procedures. The acetabular cup impactor disclosed accomplishes accurate implant orientation and implant fixation through a limited surgical exposure.

An acetabular component, such as a press fit shell, is implanted following preparation of the acetabulum. An impaction device is provided that allows for impaction of the acetabular component with the hip reduced or articulated in order to fully seat a press fit acetabular component into the acetabulum. In hip arthroplasty, the hip is accessed through an incision adequate to expose the trochanteric fossa and allow resection of the femoral neck and removal of the femoral head and neck segment. The femoral canal is accessed through the trochanteric fossa and trochanteric region. Reamers, rasps and other devices as are known to those skilled in the art are used to prepare the proximal femur to receive a femoral implant by a sequence of reaming and broaching steps. Once prepared, the intramedullary canal and retained area of the femoral neck and trochanteric region are used to support the acetabular cup impactor of the current invention.

### BRIEF DESCRIPTION OF THE DRAWINGS

FIG. 1 is a perspective view of the surgical incision through which the present invention is structured to be used.

FIG. 2 is an orthogonal view of cup impactor according to embodiment of the present invention.

FIG. 3 is a perspective view of the acetabular cup impactor, femoral broach and acetabular cup superimposed on a femur according to embodiment of the present invention.

FIG. 4 is a schematic view of a femoral broach.

FIGS. 5 and 6 are exploded views of cup impactor according to embodiment of the present invention.

FIGS. 7, 8 and 9 are cross section views of implant attachment assembly according to embodiment of the present invention.

### DETAILED DESCRIPTION OF THE INVENTION

As described above, the present invention is applicable to orthopedic surgical procedures for total hip arthroplasty; optionally the invention may be used in resurfacing hip arthroplasty. Optionally, the cup impactor of this invention may be used with an attachable alignment guide to aid in aligning and orienting the acetabular shell. Optionally, the cup impactor of this invention may be used with an attachable surgical navigational tracker to aid in aligning and orienting the acetabular shell.

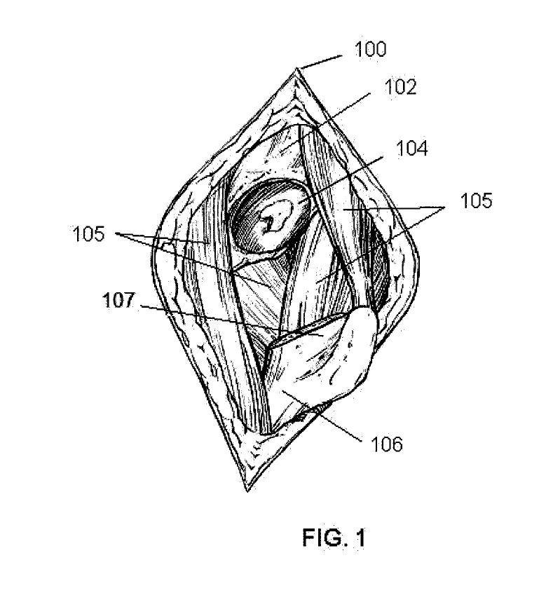

Referring to FIG. 1, there is depicted a surgical incision 100 for a less invasive total hip arthroplasty. The muscles and soft tissues spanning the hip joint are exposed and either bluntly dissected along muscle fibers or separated along muscle boundaries. Optionally, select muscles may be taken down to increase surgical exposure and access to the hip joint. Anatomy of interest to this embodiment of the invention includes the pelvis 102, the acetabulum 104, the femur 106, the joint capsule (not shown) and the muscles 105 and ligaments spanning the hip joint. The femoral head is resected at the base of the femoral neck 107 as shown in FIG. 1 to provide access to the medullary canal to prepare the canal to receive a femoral hip stem. In total hip arthroplasty, the articular surfaces of the proximal femur and the acetabulum are resurfaced. In general, after resecting the femoral head, the femur is prepared by reaming and broaching to prepare the femoral canal to receive a hip stem implant and femoral head implant there on. Alternatively, the femoral head may be sculpted to receive a resurfacing implant structured to fit over the prepared femoral head, this representing another embodiment of the present invention to place an implant onto a prepared bone surface. The acetabulum is generally prepared by reaming a hemispherical cavity to receive an acetabular cup.

In traditional total hip arthroplasty the surgical exposure generally ranges between eight and twelve inches in length and may result in extensive trauma to the soft tissues surrounding the hip joint. In minimally invasive total hip surgery, the incision 100 is typically two to four inches in length as shown in FIG. 1. While this is a typical length for a minimally invasive surgical incision, there may be some variation due to patient physiology, surgeon preferences, and/or other factors. The surgical approach involves separating the gluteus maximus muscle through blunt dissection to gain access to the hip joint capsule and the trochanteric fossa. Muscle disruption is usually limited to release of the piriformis tendon at the trochanteric fossa. It should be noted that there are variations to the surgical approaches described that are known to someone skilled in the art.

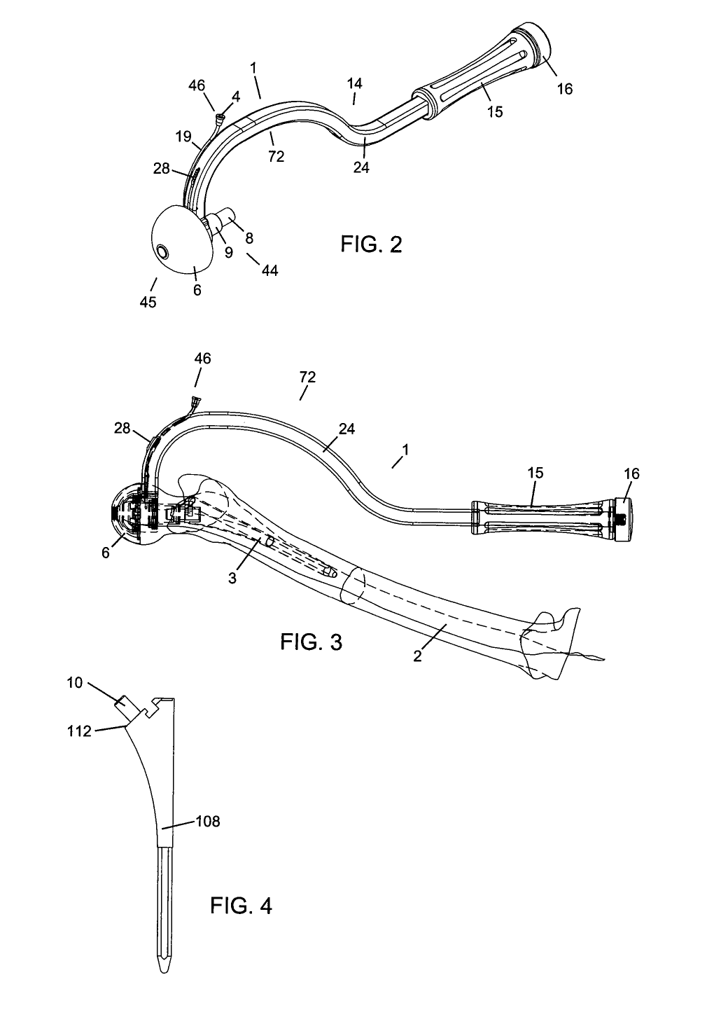

Referring now to FIG. 2, The impactor 1 having a first end and a second end. The first end having a strike plate 16 structured to receive mallet blows to impact acetabular shell 6. The second end structure to receive acetabular shell 6. Impactor 1 generally includes a handle 14, a distraction assembly 44, an implant attachment assembly 45 and a pressure input assembly 46.

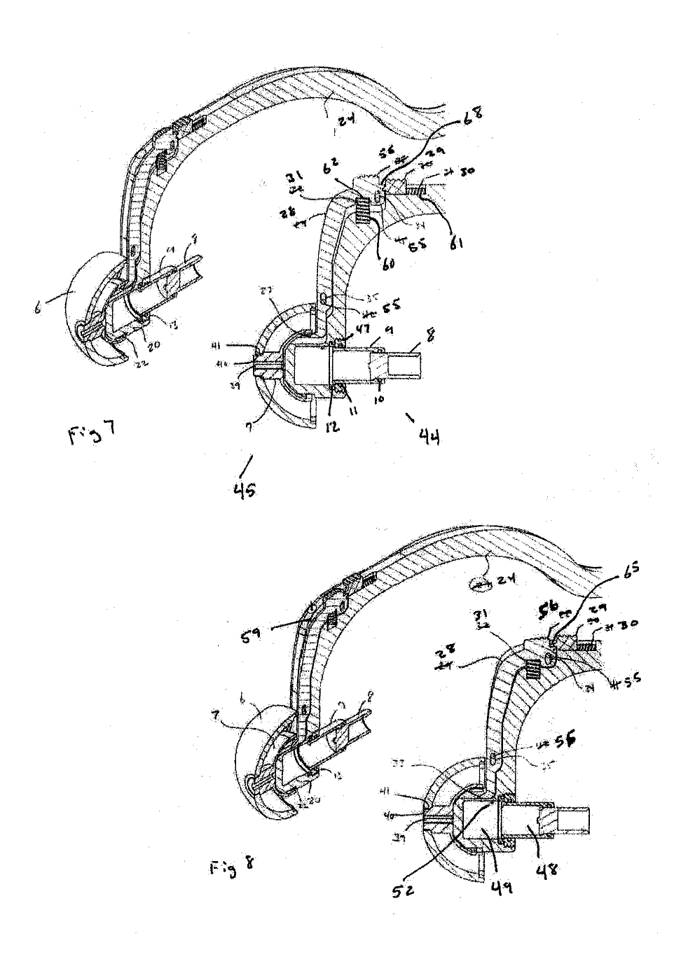

Handle 14 includes a handle shaft 24 having a grip 15 thereon, grip 15 being of a size and shape for grasping in a hand to stabilize impactor 1. Strike plate 16 generally covers surface of handle 1 first end and is joined to handle shaft 24 such that mallet blows applied to strike plant 16 are transferred to handle shaft 24. Implant attachment assembly 45 is joined to handle shaft 24 such that mallet blows applied to strike plate 16 are transferred to acetabular shell 6. Referring to FIG. 6, handle shaft 24 includes external thread 53 to threadably receive internal thread 54 of strike plate 16.

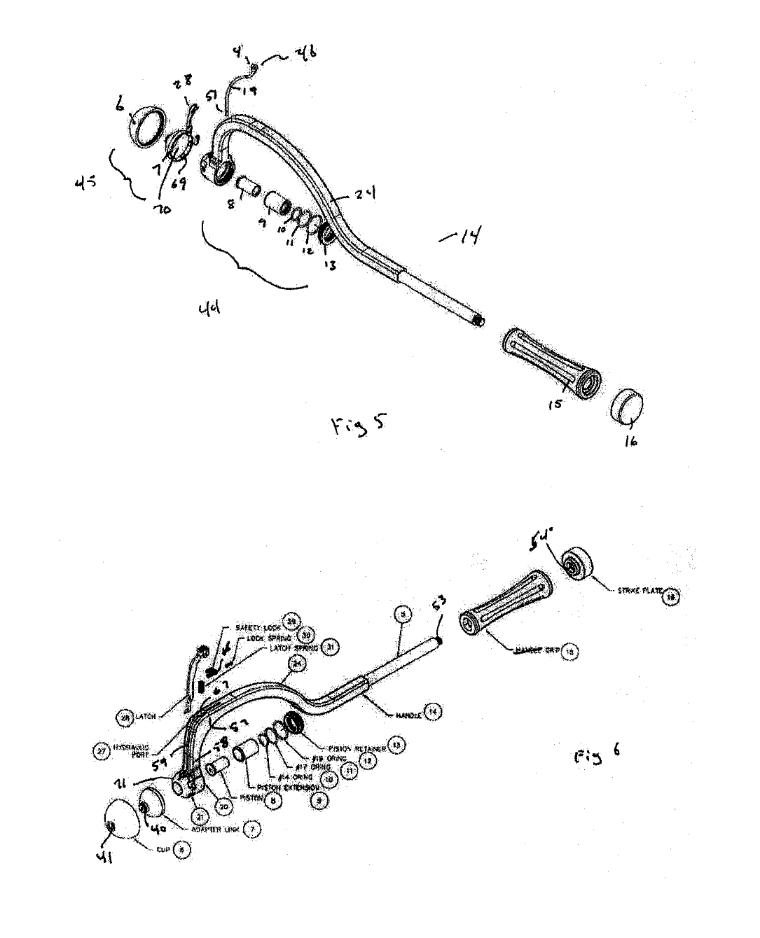

Distraction assembly 44 includes a piston 8 and a piston extension 9. Referring to FIGS. 2, 3 and 4, piston 8 is structured to slidably broach post 110. Broach 108 is supported within femur 2. Distraction assembly 44 is structured to receive pressure input assembly 46 to provide pressure to elongate piston 8 and piston extension 9 as described in greater detail hereinafter. Syringe pump (not shown) or similar hydraulic or pneumatic pressure source connected to pressure input assembly 46 to pressurize distraction assembly 44.

Implant attachment assembly 45 is structured to releasably receive acetabular shell 6 and includes latch 28 to activate lock to secure adaptor link 7 as described in greater detail hereinafter.

Pressure input assembly 46 includes Luer Lock 4 for sealable connection to syringe pump (not shown) and elongated tube 19. Elongated tube 19 sealably received by distraction assembly 44 as described in greater detail hereinafter.

Turning now to FIG. 5, second end of handle 1 is structured to slidably receive piston extension 9 therein retained by piston retainer 13. Piston extension 9 is structured to slidably receive piston 8. Distraction assembly 44 includes o-rings 10, 11 and 12 sealing interfaces between piston 8 and piston extension 9, piston extension 9 and piston retainer 13, and piston retainer 13 and handle shaft 24, respectively, as illustrated in cross section view in FIG. 7. Piston retainer 13 is structured to slidably receive piston extension 9 and to be assembled into handle shaft 24 by threaded interface 47.

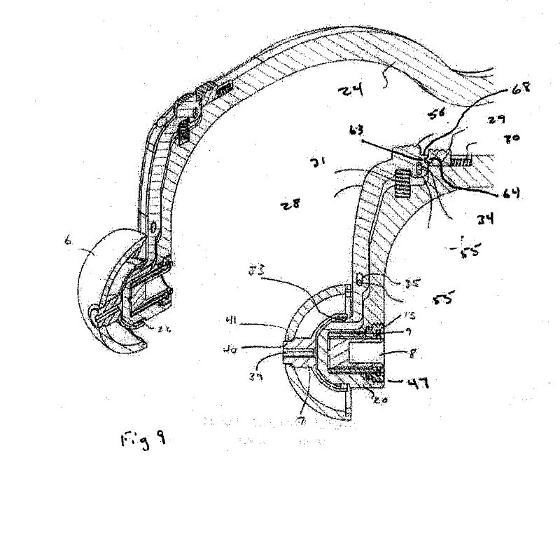

Referring to FIGS. 5 and 8, pressure input assembly includes attachment end 51 slidably and sealably received in receiving hole (not shown) in handle shaft 24. The receiving hole is in communication with handle shaft cylinder 49 via port 52, which is in communication with piston extension cylinder 49.

Implant attachment assembly 45, shown in cross section in FIGS. 6, 7, 8 and 9, includes adaptor link 7 structured to be slidably and lockably received by handle shaft 24, and structured to be assembled with acetabular shell 6. Implant attachment assembly 45 further includes latch 28, safety lock 29, lock spring 30, latch spring 31, and retaining pin 55, each of which is assembled into handle shaft 24.

Adaptor link 7 includes external thread 40 sized to be threadably received by threaded receiving hole 41 in acetabular shell 6. Adaptor link 7 being one of a set of adaptor links (not shown) of various lengths as appropriate for the size range of acetabular shells typically included in a total hip implant kit. Optionally, adaptor link set (not shown) may include various thread 40 sizes as appropriate for assembly with acetabular shells generally available. Optionally, adaptor link set may include adaptor links structured for assembly with generally available acetabular shells structured with fasteners other than threaded fasteners, for example bayonet mounts, expanding collets, or snap fits.

Assembly of latch 28, safety lock 29, lock spring 30, latch spring 31, retaining pin 55 and handle shaft 24 is as follows. Latch spring 31 is placed into receiving hole 60. Lock spring 30 is placed into receiving hole 61. Safety lock 29 is slidably received in slot 67 retained therein by tabs 66 slidably received in grooves 65 and by latch 28. Latch 28 is slidably received in slot 59 and slidably retained by retaining pins 55 placed into upper receiving hole 57 and lower receiving hole 58 in handle shaft 24. Retaining pins 55 secured in place by welding, bonding, press fit, or other suitable means know to those skilled in the art. Retaining pins 55 slidably received in upper receiving slot 34 and lower receiving slot 35 in latch 28. Latch 28 thus assembled is free to slide up and down by force applied by the operator to release button 56. In an unlocked position, shown in FIG. 8, latch 28 is depressed into handle shaft 24 and retained therein by safety lock 29 tab 64 resting on latch 28 surface 68. Sliding safety lock 29 away from latch 28 releases latch 28 to slide upward to a locked position. Tab 64 engages latch 28 slot 63 thereby retaining latch in locked position. The top face of safety lock 29 tab 64 is ramped to allow slidable release of safety lock 29 by pressing on release button 56 thereby moving the latch to unlocked position. Latch spring 31 provides bias force tending to move latch 28 towards a locked position. Lock spring 30 provides bias force tending to move safety lock 29 towards engagement with latch 28.

Turning now to connecting an acetabular shell 6 to impactor 1, handle 14 second end 71 is generally cylindrical with radius leading edge and includes six bayonet slots 21 circumferentially equally spaced. Optionally, one or more bayonet slots 21 may be used or other fasteners, for example threaded fastener, slip fit, taper fit, snap fit, etc., know to those skilled in the art. Adaptor link 7 cavity 70 is structured to slidably receive handle 14 second end 71 and releasably lock thereon. Cavity 70 including six tabs 69 circumferentially equally spaced to be received by corresponding bayonet slots 21. Lower end of latch 28 includes a tab 33 positioned to close off one of the bayonet slots 21. Latch 28 unlocked position, as shown in FIG. 8, positions tab 33 deeper than bayonet slot 21 opening. Latch 28 locked position, as shown in FIG. 9, positions tab 33 within bayonet slot 21 opening to block one adaptor link 7 tab 69 from turning out of bayonet slot 21.

Adaptor link 7 is first assembled with acetabular shell 6. With latch 28 in unlocked position, adaptor link 7 is slidably received on handle 14 second end 71 and rotated to secure tabs 69 in bayonet slots 21. Safety lock 29 is slid away from latch 28 to release latch 28 to locked position.

Distraction assembly 44 is initially retracted as shown in FIG. 9. Pressure, either hydraulic or pneumatic, applied to pressure input assembly 46 deploys piston 8 and piston extension 9 to tension the joint capsule as described in more detail hereinafter. Distraction assembly 44 is shown in full distraction position in FIG. 7. Strike plate 16, shaft handle 24, adaptor link 7, piston 8 and extension piston 9 are constructed of rigid material, such as metal or carbon-carbon composite, to withstand mallet blows typical of impacting an acetabular shell. Grip 15 is constructed of metal or plastic or laminated linen material as is know by those skilled in the art.

As shown most clearly in FIG. 3, handle shaft 24 angles abruptly, generally perpendicular, away from acetabular shell 6 axis. Optionally, handle shaft 24 may angle more acutely away from acetabular shell 6 axis in a range from 45° to 90°. Curved portion 72 of handle shaft 24 is structured for co-axial alignment of acetabular shell axis, grip 15 and strike plate 16. Curve portion 72 sized and shaped to provide clearance around anatomical features of hip joint and surrounding tissues. Such abrupt angulation of shaft handle 24 is advantageous when performing hip surgery through a limited or minimal exposure as the muscles spanning the hip are preferably left intact thereby limiting the space outside of the acetabulum.

The current invention is designed to provide alignment and orientation of the acetabular shell based on the anatomy of the pelvis, femur and on the kinematics of the hip joint. This is accomplished by tissue guided surgery “TGS” as described in patents U.S. Pat. No. 6,723,102 and patent applications US 2002/0193797 and US 2003/0236523, the entireties of which are incorporated by reference. Impactor 1 is designed to attach to a femoral broach 3 supported by femur 2. In applying TGS to hip arthroplasty, orientation of acetabular shell 6 is guided by soft tissue envelope surrounding the hip joint. This envelope of tissue defines the limits of hip motion. The soft tissue capsule working in combination with muscles spanning the hip and the articular joint surfaces of the hip define hip kinematics. TGS utilizes such kinematics to first prepare the acetabulum, then to orient and place acetabular shell 6. Femur 2 is used as a reference to guide impactor 1 to orient acetabular shell 6 relative to acetabulum by using the joint capsule to properly position and orient the femur with respect to the acetabulum.

Surgical Procedure

Impactor 1 is structured for partially disassembled for cleaning and sterilization. The components of the impactor are housed in an instrument tray which is brought to the operating room sterile. The instrument tray has fixtures to hold individual components and markings to show where components are to be placed. Impactor 1 is assembled in the operating room under sterile conditions. Distraction assembly 44 is fully retracted. A syringe pump (not shown) or suitable sterile fluid pressurizing source is charged with sterile saline and attached to pressure input assembly 46.

After reaming the acetabulum and with the femoral broach in place, the appropriate size acetabular shell 6 is selected. The corresponding adaptor link 7 is selected and assembled to the shell 6. The adaptor link 7 is attached to handle shaft 24 as described above.

Impactor 1 with acetabular shell 6 attached is used to place the shell 6 into the prepared acetabulum. Acetabular shell 6 is oriented with respect to the acetabulum by properly aligning the femur with the pelvis then deploying distraction assembly 44 as previously described to tensioning joint capsule. Cup alignment may be confirmed with a mechanical alignment guide (not shown) or with a surgical navigation system and tracker (not shown).

Acetabular shell 6 is now in proper position and orientation with respect to the acetabulum. The surgeon uses a mallet (not shown) to impact acetabular shell 6 by striking the strike plate 16. Mallet blows are repeated until acetabular shell 6 is fully seated in the acetabulum. Distraction assembly 44 is retracted. Handle shaft 24 is released from adaptor link 7 as described above and removed from surgical site. Adaptor link 7 is removed from acetabular shell 6 using a hex driver (not shown) attaching to the hex drive 39. The cup is now placed in the acetabulum and the total hip arthroplasty procedure continues per the surgical technique.

While a preferred embodiment of the present invention has been described, it should be understood that various changes, adaptations and modifications may be made therein without departing from the spirit of the invention and the scope of the appended claims.

1. A system for imparting force between adjacent first and second bones to seat an implant in the second bone, the system comprising:
an implant; and
an impaction device including an attachment portion slidably receivable by a bone mount placed in the first bone, the impaction device including a handle having a curved shaft and first and second ends, said first end including a strike plate threadably received thereon and structured to receive an impaction force, said second end structured to receive said implant, wherein upon application of force to said strike plate the implant is press fit in the second bone, said force reacted by said adjacent first and second bones.
